(19) United States
(12) Patent Application Publication
(10) Pub. No.: US 2004/0220539 A1
(43) Pub. Date: Nov. 4, 2004

(54) Absorbent article with improved absorbency performance

(76) Inventors: Frank S. Glaug, Chester Springs, PA (US); John D. Litvay, Downington, PA (US)

Correspondence Address: HUNTON & WILLIAMS LLP, INTELLECTUAL PROPERTY DEPARTMENT, 1900 K STREET, N.W., SUITE 1200, WASHINGTON, DC, 20006-1109, US

(21) Appl. No.: 10/426,683

(22) Filed: May 1, 2003

(51) Int. Cl. A61F 13/15; A61F 13/20

(52) U.S. Cl. 604/367; 604/389

(57) ABSTRACT

The present invention relates generally to an absorbent article, and more particularly to a fluid transfer layer including tow fibers. Such fluid transfer layers provide improved acquisition and rewet values for the absorbent article.

### FIELD OF THE INVENTION

The present invention relates generally to an absorbent article, and more particularly to an acquisition layer for use in an absorbent article whereby the acquisition layer includes tow fibers. Such absorbent articles have improved absorbency, improved strikethrough and improved rewet values.

### BACKGROUND OF THE INVENTION

Disposable absorbent articles typically include a moisture-impervious backing sheet, an absorbent pad, and a liner sheet that contacts the body of a person wearing the article. In addition, elasticized regions are provided around the edges of the article to secure the article about the waist and legs of a wearer. Absorbent articles such as diapers typically further comprise opposed front and rear waist portions defining a waist opening, a crotch portion disposed there between, and a pair of elastically contractible leg openings along the side edges of the crotch portion. Disposable diapers having elasticized margins for placement about the legs of a wearer are disclosed in U.S. Pat. No. 4,050,462 and U.S. Pat. No. 5,092,861, the disclosures of which are incorporated by reference herein in their entirety. An absorbent article having elasticized side margins and waist band margins are described in U.S. Pat. No. 4,300,562, the disclosure of which is incorporated by reference herein in their entirety.

Despite previous advancements in the field of absorbent articles, there still is a current need to provide absorbent articles that are better able to contain urinary and fecal excretions. For instance, problems with prior diaper designs include inferior absorbency and leakage of urinary or fecal material from the article. Prolonged contact of liquid or semi-solid excreta with the skin of the wearer also is a continuing problem. For example, the moisture vapor and heat generated by the body exudates trapped within a diaper may lead to conditions adjacent to the wearer's skin that promotes skin irritation, infection, and the like.

Developing highly absorbent articles for use as disposable diapers, adult incontinence pads and briefs, and catamenial products such as sanitary napkins typically entails developing relatively thin absorbent cores or structures that can acquire, distribute and store large quantities of discharged body fluids, in particular urine. For example, absorbent structures include particulate absorbent polymers often referred to as “hydrogels,” “superabsorbents” or “hydrocolloid” materials. See, for example, U.S. Pat. No. 3,699,103, and U.S. Pat. No. 3,770,731 that disclose the use of such particulate absorbent polymers in absorbent articles. Indeed, the development of high performance diapers has been due in part to thinner absorbent cores that take advantage of the ability of these particulate absorbent polymers to absorb large quantities of discharged aqueous body fluids, typically when used in combination with a fibrous matrix. See, for example, U.S. Pat. No. 4,673,402 and U.S. Pat. No. 4,935,022, that disclose dual-layer core structures comprising a fibrous matrix and particulate absorbent polymers useful in fashioning high performance diapers.

It is known to provide thin absorbent laminates comprised of, for example, an upper and lower layers, and a central fibrous layer containing from 50% to 95% by weight SAP, and cellulose acetate tow fibers. U.S. Pat. No. 6,068,620, the disclosure of which is incorporated herein by reference in its entirety, discloses that the upper and lower layers are comprised of tissue, airlaid fluff pulp or synthetic non-woven fibrous layers. The upper and lower layers are said to assist in maintaining the integrity of the core, the laminate layered arrangement is said to minimize gel blocking, and the laminate can be folded in various configurations.

It also has been known to prepare absorbent cores comprised of cellulose acetate tow or other polymeric fibers and SAP, as described in H1565, and U.S. Pat. Nos. 5,436,066, and 5,350,370, the disclosures of each of which are incorporated by reference herein in their entirety. It was conventional to add tackifying agents, specific size fibers, or specific fibers in combination with fluff, in order to prepare the absorbent core and immobilize the SAP particles.

A disadvantage of using higher density, thinner cores is that the cores do not absorb liquid as rapidly as lower density cores, because densification of the core results in smaller effective pore size. Accordingly, it is desirable to provide a lower density layer having a larger pore size above the high density absorbent core to maintain a suitable liquid absorption rate, and to increase the rate of uptake of liquid discharged onto the absorbent article. The low density layer is typically referred to as an acquisition layer, and acquisition/distribution layer (ADL).

The acquisition layer portion of a disposable diaper typically is comprised of a carded synthetic staple fiber web that is thermally bonded, latex bonded, or point bonded. Typical staple fibers for acquisition layers are crimped polyester (PET) or polypropylene fibers that have a size of 6 to 15 denier and a length of at least 40 mm. The acquisition layer is formed, bonded and slit as a homogenous rolled good on a dedicated nonwoven textile production line. The slit roll of acquisition layer material is subsequently unrolled onto the diaper converting line where it is affixed on top of the absorbent core and below the topsheet.

One embodiment of a structure having an acquisition layer and a distribution layer (an “ADL”) typically found on die-cut feminine hygiene pads is an airlaid cellulose web bonded with an aqueous binder resin that has been dried and cured. Airlaid materials typically retain up to 16 g of fluid per gram of material against gravity under negligible load. Thus, an ADL can acquire a surge of fluid within the absorbent product until the superabsorbent particles in the absorbent core can absorb the retained fluid out of the airlaid cellulose ADL and into final storage containing superabsorbent particles.

An example of a conventional airlaid cellulose material is Vicell 6002 (Buckeye Technologies Inc., Memphis Tenn.), which is a 105 gsm (grams per square meter) airlaid cellulose non-woven bonded with a vinyl acetate binder resin. Vicell 6002 is prepared by spraying an aqueous emulsion of the vinyl acetate binder resin onto the airlaid cellulose web followed by drying and curing in a hot air oven. It is used commercially in an ADL for feminine hygiene pads.

The disadvantage of certain commercially available airlaid cellulose structures is that they may collapse under normal use. This typically occurs when the structure is compressed by the weight of the wearer and particularly when the structure becomes wet. This structural collapse significantly reduces the fluid acquisition rate of the absorbent product and thus increases the chance of leakage. When a completely or partially fluid saturated airlaid cellulose structure collapses, the fluid escapes from the ADL and the product feels wet against the wearer's skin.

A fluid acquisition layer stated to overcome some of these disadvantages is disclosed in U.S. Pat. No. 6,479,415, to Erspamer, et al., (“Erspamer”). Erspamer discloses an acquisition layer that includes synthetic fibers where the fibers are latex bonded. The ADL includes two layers, the top layer comprising the latex bonded synthetic fibers, and the bottom layer including latex and/or thermal bonded cellulose fibers.

There are a number of documents describing absorbent foams useful as an ADL, where the foams have been made from High internal Phase Emulsions (hereafter referred to as “HIPE”), or hydrophilic, flexible, open-celled foam such as a melamine-formaldehyde foam (e.g., BASOTEC™ made by BASF). See, for example, U.S. Pat. Nos. 5,147,345, 5,260,345, 5,268,224, 5,318,554, 5,331,015, 5,352,711, 5,550,167, 5,632,737, 5,692,939, 5,786,395, and 5,851,648. These absorbent HIPE foams provide desirable fluid handling properties, including: (a) relatively good wicking and fluid distribution characteristics to transport the imbibed urine or other body fluid away from the initial impingement zone and into the unused balance of the foam structure to allow for subsequent gushes of fluid to be accommodated; and (b) a relatively high storage capacity with a relatively high fluid capacity under load, i.e. under compressive forces. These HIPE absorbent foams are also sufficiently flexible and soft so as to provide a high degree of comfort to the wearer of the absorbent article; some can be made relatively thin until subsequently wetted by the absorbed body fluid.

The use of tow fibers in acquisition layers also is known and described, for example, in U.S. Pat. Nos. 4,360,022, 6,245,961, 6,417,427, and 6,511,566, the disclosures of each of which is incorporated by reference herein in their entirety. These documents describe, for example, thermally bonding the tow fibers to form a fibrous material layer, or to facilitate forming various zones in the acquisition layer.

As is apparent from the foregoing, each of these documents presents a variety of mechanisms for improving absorbency in absorbent garments. However, all of these proposed mechanisms are deficient in terms of effectiveness, low product quality, mechanical complexity in design, and/or associated cost inefficiencies.

The description herein of the various known products, methods, and apparatus and their attendant disadvantages is in no way intended to limit the scope of the present invention, or to imply that the present invention does not include some or all of the various elements of the products, methods, and apparatus in one form or another. Indeed, various embodiments of the invention may be capable of overcoming some of the disadvantages noted herein, while still retaining some or all of the various elements of the known products, methods, and apparatus in one form or another.

### SUMMARY OF THE INVENTION

It would be desirable to provide an absorbent garment having an improved ability to retain fluids and consequently, to prevent leakage. It also would be desirable to provide an absorbent article that exhibits an improved rate of acquisition of fluid, and a reduced amount of fluid rewet.

It therefore is a feature of an embodiment of the invention to provide an absorbent garment having an improved ability to acquire and retain fluids, especially in areas of the core where fluid retention is needed most. It is an additional feature of an embodiment of the invention to provide an absorbent garment that includes a transfer layer comprised of a fibrous material that has the improved absorbency characteristics noted above, and that is comfortable to wear.

These and other features of the invention can be achieved by an absorbent article including a top sheet, a back sheet an absorbent core disposed between the top sheet and the back sheet, and a transfer layer disposed between the topsheet and the absorbent core, whereby the transfer layer includes tow fibers and the absorbent article has a third insult strikethrough time of less than about 35 seconds, and a third insult rewet value of less than about 30 grams.

In accordance with an additional feature of an embodiment of the invention, there is provided a method of making an absorbent article that includes providing a top sheet material and a back sheet material. The method also include preparing an absorbent core and disposing the absorbent core between the top sheet material and the back sheet material. The method further includes preparing a transfer layer comprised of tow fibers, and disposing the transfer layer between the top sheet material and the absorbent core to form an absorbent article that has a third insult strikethrough time of less than about 35 seconds, and a third insult rewet value of less than about 30 grams.

These and other features and advantages of the preferred embodiments will become more readily apparent when the detailed description of the preferred embodiments is read in conjunction with the attached drawings.

### BRIEF DESCRIPTION OF THE DRAWINGS

FIG. 1 is a partially cut-away view of an embodiment of the present invention, shown with elastic members fully stretched in the main portion of the garment;

FIG. 2 is a cross-section of the article of FIG. 1, taken along line A-A.

FIGS. 3a and 3b are cross-sectional views of the absorbent core and transfer layer containing tow fibers;

FIGS. 4a, 4b, and 4c are cross-sectional views of the absorbent core and transfer layer containing tow fibers;

FIG. 5 is a view of the apparatus used in the examples to measure the acquisition time of transfer layers;

FIG. 6 is a chart showing the relationship between transfer layer basis weight and acquisition time;

FIG. 7 is a chart showing the relationship between transfer layer fiber denier and acquisition time;

FIG. 8 is a chart showing a comparison of strikethrough time for various transfer layers;

FIG. 9 is a chart showing a comparison of rewet values for various transfer layers;

FIG. 10 is a chart showing the relationship between transfer layer basis weight and rewet values;

FIG. 11 is a chart showing the relationship between transfer layer fiber denier and rewet values;

### DETAILED DESCRIPTION OF THE PREFERRED EMBODIMENTS

As used herein, the terms “absorbent garment,” “absorbent article” or simply “article” or “garment” refer to devices that absorb and contain body fluids and other body exudates. More specifically, these terms refer to garments that are placed against or in proximity to the body of a wearer to absorb and contain the various exudates discharged from the body. A non-exhaustive list of examples of absorbent garments includes diapers, diaper covers, disposable diapers, training pants, feminine hygiene products and adult incontinence products. Such garments may be intended to be discarded or partially discarded after a single use (“disposable” garments). Such garments may comprise essentially a single inseparable structure (“unitary” garments), or they may comprise replaceable inserts or other interchangeable parts.

The present invention may be used with all of the foregoing classes of absorbent garments, without limitation, whether disposable or otherwise. The embodiments described herein provide, as an exemplary structure, a diaper for an infant, however this is not intended to limit the claimed invention. The invention will be understood to encompass, without limitation, all classes and types of absorbent garments, including those described herein. Preferably, the absorbent core is thin in order to improve the comfort and appearance of a garment.

Throughout this description, the expressions “upper layer,” “lower layer,” “above” and “below,” which refer to the various components included in the absorbent core units of the invention (including the layers surrounding the absorbent core units) are used merely to describe the spatial relationship between the respective components. The upper layer or component “above” the other component need not always remain vertically above the core or component, and the lower layer or component “below” the other component need not always remain vertically below the core or component. Indeed, embodiments of the invention include various configurations whereby the core is folded in such a manner that the upper layer ultimately becomes the vertically highest and vertically lowest layer at the same time. Other configurations are contemplated within the context of the present invention.

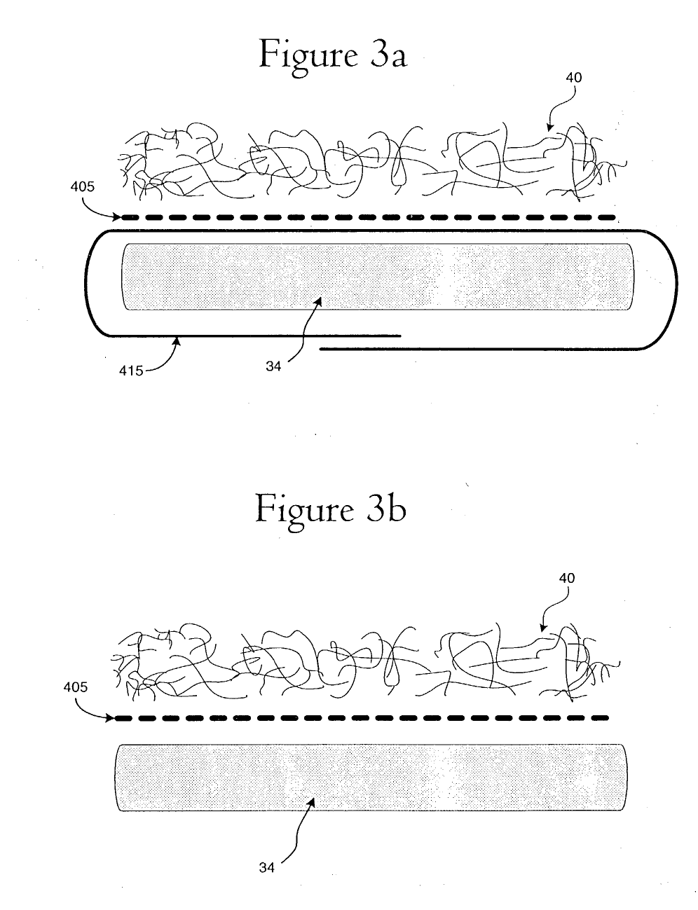

In addition, upper and lower layer refers to the ultimate configuration of the absorbent core, a preferred cross section of which is illustrated in FIG. 3a. The upper and lower layers may be comprised of the same material folded over the absorbent core 34. Thus, in essence only one layer of material is used in forming the core.

The term “component” can refer, but is not limited, to designated selected regions, such as edges, corners, sides or the like; structural members, such as elastic strips, absorbent pads, stretchable layers or panels, layers of material, or the like; or a graphic. The term “graphic” can refer, but is not limited, to any design, pattern, indicia or the like.

Throughout this description, the term “disposed” and the expressions “disposed on,” “disposing on,” “disposed in,” “disposed between” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

Throughout this description, the terms “top sheet” and “back sheet” denote the relationship of these materials or layers with respect to the absorbent core. It is understood that additional layers may be present between the absorbent core and the top sheet and back sheet, and that additional layers and other materials may be present on the side opposite the absorbent core from either the top sheet or the back sheet.

Throughout this description, the expression “tow fibers” relates in general to any continuous fiber. Tow fibers typically are used in the manufacture of staple fibers, and preferably are comprised of natural and/or synthetic thermoplastic polymers. Usually, numerous filaments are produced by melt extrusion of the molten polymer through a multi-orifice spinneret during manufacture of staple fibers from synthetic thermoplastic polymers in order that reasonably high productivity may be achieved. The groups of filaments from a plurality of spinnerets typically are combined into a tow which is then subjected to a drawing operation to impart the desired physical properties to the filaments comprising the tow. Tow as used in the context of the present invention also encompasses modified tow fibers that have been either surface or internally modified (chemically or otherwise) to improve various desired properties of the fibers (e.g., wicking, etc.).

The term “strikethrough” is used herein to denote the amount of time it takes for liquid to pass through the material being tested. Strikethrough is a measure of the fluid acquisition properties of the material. Strikethrough is measured in accordance with the test procedures defined below. Unless indicated otherwise, strikethrough values are reported herein in seconds.

The term “rewet” is used herein to mean retransmission of liquid from the absorbent core to the body or wearer side of the topsheet when the disposable absorbent article is in use. Rewet therefore is a measure of the absorbent article's fluid retention capabilities under load. Low rewet means low retransmission of liquid from the fluid transport layer and/or absorbent core to the body or wearer side of the topsheet. The rewet property of an absorbent article is determined by the procedure outlined in the test procedures section below. Unless indicated otherwise, rewet values are reported herein in grams.

The present invention provides an absorbent article, as well as a method of preparing the same and a method of using the absorbent article. The absorbent article has superior properties of absorbency, leakage protection and/or skin wellness, as well as being aesthetically pleasing. These properties are a feature of the fluid transfer layer's ability to rapidly acquire fluid, and then to retain the fluid and redistribute it to the absorbent code while under load.

Those skilled in the art recognize that a material that has a good strikethrough characteristic typically has poor fluid retention (e.g., rewet) characteristics. This is because good strikethrough is a measure of how fast the material can acquire fluid. Materials that can quickly acquire fluid typically are quite porous, have good wicking properties, and/or have well defined fluid flow channels. Consequently, these materials by design typically lose their fluid as quickly as they acquire it, which translates to poor rewet, or poor fluid retention.

The present invention relates generally to absorbent articles, and in particular to an absorbent article that contains a top sheet, a back sheet, an absorbent core disposed at least partially between the top sheet and the back sheet, and a transfer layer disposed between the top sheet and the absorbent core. The transfer layer is comprised at least in part of tow fibers and the absorbent article has a third insult strikethrough time of less than about 35 seconds, and a third insult rewet value of less than about 30 grams.

The invention also relates in general to a method of making an absorbent article that includes providing a top sheet material and a back sheet material. The method also includes preparing an absorbent core and disposing the core between the top sheet and the back sheet. The method further includes preparing a transfer layer comprised at least in part of tow fibers, and disposing the transfer layer between the top sheet and the absorbent core to form an absorbent article that has a third insult strikethrough time of less than about 35 seconds, and a third insult rewet value of less than about 30 grams. Adhesive may optionally be used to assist in adhering the transfer layer to the absorbent core, or a component of the absorbent core (e.g., a tissue or non-woven layer(s) surrounding the core, a tissue or non-woven layer(s) surrounding the transfer layer, and the like).

The absorbent article of the invention preferably has a front waist region, a rear waist region and a crotch region positioned between the front and rear waist regions. The front waist region and rear waist region can be associated with one another to form a waist opening, and two leg openings. Those skilled in the art recognize that “front” and “rear” in the context of the invention denote for clarity purposes only the front and rear of a user, and that the absorbent article could be reversed whereby the previously described “front” portion becomes the rear portion, and vice versa.

Leg elastics preferably are provided along the leg openings for securely holding the leg openings against the thighs of the wearer to improve containment and fit. A fastening system, either resealable or permanent, preferably holds the absorbent article around the wearer's waist. The fastening system assists in associating the front waist region with the rear waist region. A pair of stand-up leg gathers or waist containment flaps may be attached to or formed from the body's side surface of the top sheet.

In the present invention, the absorbent article preferably has a third insult strikethrough of less than about 35 seconds, and a third insult rewet of less than about 30 grams. More preferably, the absorbent article has a third insult strikethrough of less than about 30 seconds, and a third insult rewet of less than about 25 grams, and more preferably, third insult strikethrough of less than about 25 seconds, and a third insult rewet of less than about 22 grams. Most preferably, the, the absorbent article has a third insult strikethrough of less than about 20 seconds, and a third insult rewet of less than about 20 grams. Although described together, the preferred absorbent articles need not have the same combination of preferred strikethrough and rewet, thus enabling an absorbent article having, say, a third insult strikethrough of less than about 35 seconds, and a third insult rewet of less than about 20 grams.

It is preferred in the present invention to characterize the absorbent articles by their third insult strikethrough and rewet values. This is believed to be because many materials will have comparable first insult strikethrough and rewet values, but materials that prevent leakage will differentiate from the other materials at the second and third insult values. In addition, first insult strikethrough and rewet data does not provide much information about an absorbent article in a stressed condition (e.g., at the upper end of the capability of the article). Thus, first insult strikethrough and rewet values are not necessarily good predictors of materials that will have good third insult strikethrough and rewet. In addition, it is not practical to change an absorbent article after the first insult, and in the evening, three or more insults typically occur prior to changing the absorbent article. Consequently, absorbent articles having good third insult strikethrough and rewet will be more suitable for extended use absorbent articles. Moreover, it is believed that absorbent garments that have low third insult rewet values keep the skin dryer immediately after use, and since dry skin helps keep the skin healthy, consumers recognize low rewet as a benefit. Finally, it is believed that absorbent garments having low third insult strikethrough can improve the leakage performance of the absorbent article.

The absorbent article now will be described in more detail with reference to the attached Figures. For clarity, features that appear in more than one Figure have the same reference number in each Figure.

FIG. 1 is a partially cut away depiction of an exemplary embodiment of a garment 10 of the present invention. The embodiment shown in FIG. 1 is an infant's diaper, however, this depiction is not intended to limit the invention. The garment 10 of FIG. 1 is depicted in a flattened position, with the various elastic components depicted in their extended position for clarity. In the flattened position, the garment 10 generally has an hourglass shaped structure, but it may also have a rectangular or trapezoidal shape.

As used herein, the longitudinal axis 100 of the garment is the dimension of the garment corresponding to the front-to-rear dimension of the user, and the lateral axis 102 of the garment is the dimension corresponding to the side-to-side dimension of the user.

In use, an embodiment of the absorbent article of the invention is a pant-like garment 10 having a waist-encircling region and a crotch region. The waist-encircling region may comprise a front region 12, corresponding to the front of a wearer's body, and a rear region 14, corresponding to the back of a wearer's body, that are joined together at or near their lateral edges 28, causing the longitudinally distal edges 18 of the garment 10 to form the perimeter of a waist opening. The crotch region 16 extends between the front and rear regions 12, 14, and the crotch edges 20 form the perimeter of a pair of leg openings.

The front and rear regions 12, 14 may be joined to one another by permanent seams (not shown) or by releasable fasteners 22. The releasable fasteners 22 may comprise an adhesive tape, a mechanically interlocking fastener, such as a hook and loop fastener 24 or a button or snap, or any other suitable releasable fastening device (e.g., string, twist tie, etc.). The garment also may comprise a target surface 26 associated with a surface of the garment 10, that is selected to interact with the releasable fasteners 22 to provide them with the grip necessary to hold the garment 10 together. While the embodiment depicted in FIG. 1 shows the releasable fasteners 22 being located in the rear region 14, and the target surface 26 being in the front region 12, skilled artisans will recognize that these locations may be reversed. While the embodiment shown in FIG. 1 shows a hook and loop fastener 22, 24, those skilled in the art will appreciate that any fastening mechanism 22 can be used in the present invention. The selection and manufacture of permanent side seams, releasable fasteners 22, and target surfaces 26 is known in the art, and a skilled artisan can produce such structures and devices without undue experimentation.

The garment 10 preferably comprises a top sheet 30, and a back sheet 32, which may be substantially coterminous with the top sheet 30. When the garment 10 is being worn, the top sheet 30 faces the wearer's body, and the back sheet 32 faces away from the wearer. A fluid transfer layer 40 preferably is located between the top sheet 30 and the back sheet 32, whereby the transfer layer is comprised at least of tow fibers. The fluid transfer layer 40 may extend from the front region 12, through the crotch region 16, and into the rear region 14, and can be attached to the garment 10 in at least one of the front region 12 and the rear region 14. A particularly preferred configuration of the invention contemplates one in which the lateral width and longitudinal length of the fluid transfer layer 40 are slightly smaller than the respective width and length of the underlying absorbent core 34.

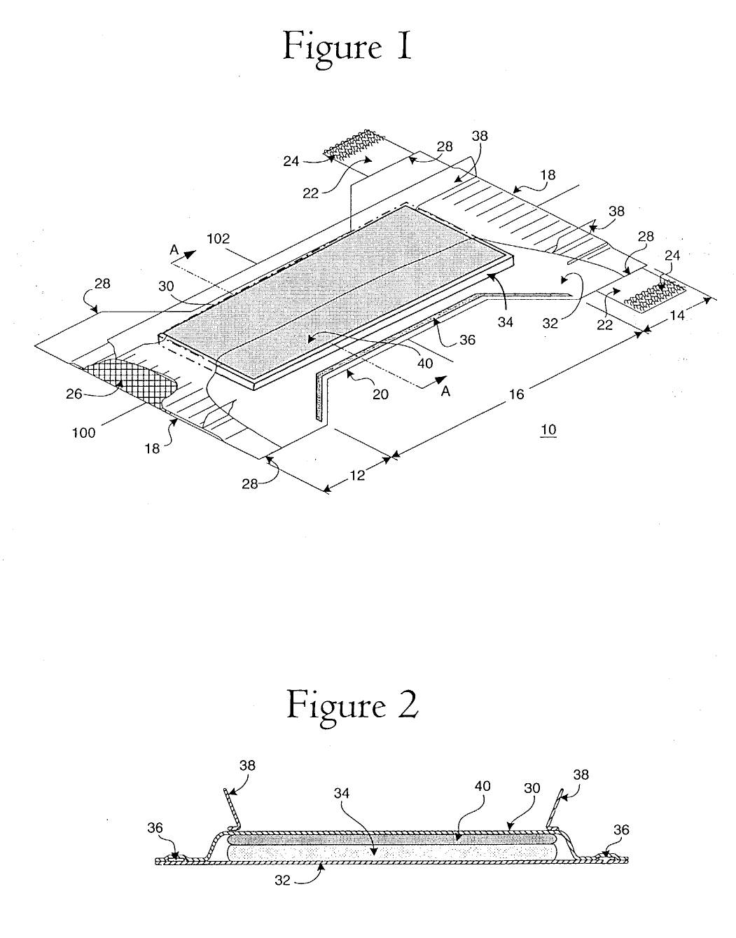

An absorbent core 34 preferably is disposed between at least a portion of the top sheet 30 and back sheet 32. Preferably, the absorbent core 34 is disposed between at least a portion of the back sheet 32 and fluid transfer layer 40. The preferred relative positions of the top sheet 30, back sheet 32, absorbent core 34 and fluid transfer layer 40 may be seen in FIG. 2, which is a cross-sectional view of the garment 10 of FIG. 1 as seen from reference line AA.

Preferred embodiments of the present invention may further comprise various additional features. One or more pairs of leg elastics 36 may extend adjacent the crotch edges 20. The garment 10 also may comprise one or more waste containment systems, such as a pair of standing leg gathers 38. The standing leg gathers 38 preferably extend longitudinally between the front region 12 and the rear region 14 along opposite sides of the garment's longitudinal axis 100. The standing leg gathers 38 are shown in FIG. 2 as being comprised of a ribbon-type material disposed above the absorbent core 34. Standing leg gathers 38 may be comprised of different materials, such as a 3-piece top sheet, as is known in the art, and may be disposed anywhere along the top sheet 30, either above the absorbent core 34, on the side edges of the core 34, or laterally outward of the absorbent core.

The various parts of the garment 10 preferably are associated with one another to form a structure that preferably maintains its shape during the useful life of the garment 10. As used herein, the term “associated” encompasses configurations whereby a first part is directly joined to a second part by affixing the first part directly to the second part, by indirectly joining the first part to the second part through intermediate members, and by fixing the relative positions of various parts by capturing parts between other parts. Those skilled in the art will appreciate that various methods or combinations of methods may be used to securely join the parts of the garment 10.

The top sheet 30 and back sheet 32 may be constructed from a wide variety of materials known in the art. The invention is not intended to be limited to any specific materials for these components. The top sheet 30 and back sheet 32 can be shaped and sized according to the requirements of each of the various types of absorbent garment, or to accommodate various user sizes. In an embodiment of the invention in which the garment 10 is a diaper or an adult incontinence brief, the top sheet 30, back sheet 32, or both, may have an hourglass shape, as seen in FIG. 1, or may have a rectangular, trapezoidal, “T” shape, or other shape. In an embodiment of the invention in which the garment 10 is a feminine hygiene product, the top sheet 30, back sheet 32, or both, may have a rectangular or ovate shape, and may have tabs or “wings.”

The back sheet 32 generally is made of any suitable pliable liquid impervious material known in the art or later discovered. Typical back sheet materials include films of polyethylene, polypropylene, polyester, nylon, and polyvinyl chloride and blends of these materials. For example, the back sheet 32 may be made of a polyethylene film having a thickness in the range of 0.02-0.04 mm. The back sheet 32 may be pigmented with, for example, titanium dioxide, calcium carbonate, and other white pigments, to provide the garment 10 with a pleasing color or to render the back sheet 32 opaque enough that exudates being contained by the garment 10 are not visible from outside the garment. In addition, back sheet 32 may be formed in such a manner that it is opaque, for example, by using various inert components in the polymeric film and then biaxially stretching the film. Other back sheet materials will be readily apparent to those skilled in the art. The back sheet 32 preferably should have sufficient liquid imperviousness to prevent any leakage of fluids through the back sheet 32. The required level of liquid imperviousness may vary between different locations on the garment 10.

The back sheet 32 may further comprise separate regions having different properties. In a preferred embodiment, portions of the back sheet 32 are air-permeable to improve the breathability, and therefore comfort, of the garment 10. The different regions may be formed by making the back sheet 32 of a composite of different sheet materials, chemical treatment, heat treatment, or other processes or methods known in the art. Some regions of the back sheet 32 may be fluid pervious. In one embodiment of the invention, the back sheet 32 is fluid impervious in the crotch 12, but is fluid pervious in portions of the front and rear regions 12, 14. The back sheet 32 may also be made from a laminate of overlaid sheets of material.

The back sheet 32 may be covered with a fibrous, non-woven fabric such as is disclosed, for example, in U.S. Pat. No. 4,646,362 issued to Heran et al., which is hereby incorporated by reference in its entirety and in a manner consistent with the present application and invention. Materials for such a fibrous outer liner include a spun-bonded non-woven web of synthetic fibers; a non-woven web of cellulosic fibers, textile fibers, or a blend of cellulosic and textile fibers; a spun-bonded non-woven web of synthetic fibers mixed with cellulosic, pulp fibers, or textile fibers; and melt blown thermoplastic fibers or mixtures of such thermoplastic fibers with cellulosic, pulp or textile fibers. These materials are well known and readily available in the art.

The moisture-pervious top sheet 30 may be made of any suitable relatively liquid-pervious material currently known in the art or later discovered that permits passage of a liquid there through. Examples of suitable top sheet materials include non-woven spun-bonded or carded webs of polypropylene, polyethylene, nylon, polyester and blends of these materials, perforated, apertured, or reticulated films, and the like. Non-woven materials are exemplary because such materials readily allow the passage of liquids to the underlying absorbent core 34.

The top sheet 30 preferably comprises a single-ply non-woven material that may be made of carded fibers, either adhesively or thermally bonded, perforated or apertured film, spun-bonded fibers, or water entangled fibers, which generally weigh from 0.3-0.7 oz./sq. yd. and have appropriate and effective machine direction and cross-machine (transverse) direction strength suitable for use as a top sheet material for the given application. The present invention is not intended to be limited to any particular material for the top sheet 30, and other top sheet materials will be readily apparent to those skilled in the art.

The top sheet 30 may further comprise several regions having different properties. In one embodiment of the present invention, the laterally distal portions of the top sheet 30 are preferably substantially fluid impervious and hydrophobic, while the remainder of the top sheet 30 is hydrophilic and fluid pervious. Different top sheet properties, such as fluid perviousness and hydrophobicity, may be imparted upon the top sheet 30 by treating the top sheet 30 with adhesives, surfactants, or other chemicals, using a composite of different materials, or by other means. Alternatively, the different properties can be achieved by making the top sheet from three separate components, a central, fluid pervious portion, and two lateral fluid impervious portions that can also serve to form standing leg gathers. Such a configuration is described in, for example, U.S. Pat. No. 6,068,620, the disclosure of which is incorporated by reference herein in its entirety. The top sheet 30 also may be made from a laminate of overlaid sheets of material. The top sheet 30 also may be treated in specific areas like the crotch region, with skin wellness ingredients like aloe and vitamin E.

As noted elsewhere herein, the top sheet 30 and back sheet 32 may be substantially coterminous, or they may have different shapes and sizes. The particular design of the top sheet and back sheet may be dictated by manufacturing considerations, cost considerations, and performance considerations. Preferably, the top sheet 30 is large enough to completely cover the absorbent core 34, and the back sheet 32 is large enough to prevent leakage from the garment 10. The design of top sheets 30 and back sheets 32 is known in the art, and a skilled artisan will be able to produce an appropriate top sheet 30 and an appropriate back sheet 32 without undue experimentation, using the guidelines provided herein.

The top sheet 30 and the back sheet 32 may be associated with one another using a variety of methods known in the art. For example, they may be thermally, ultrasonically, chemically, or thermal mechanically bonded to one another. They also may be joined using lines of hot melt adhesive or mechanical fasteners, such as thread, clips, or staples. In one embodiment, a hydrophilic adhesive, such as CYCLOFLEX™ as sold by National Starch, located in Bridgewater, N.J., is used to join the top sheet 30 to the back sheet 32. The particular joining method may be dictated by the types of materials selected for the top sheet 30 and back sheet 32.

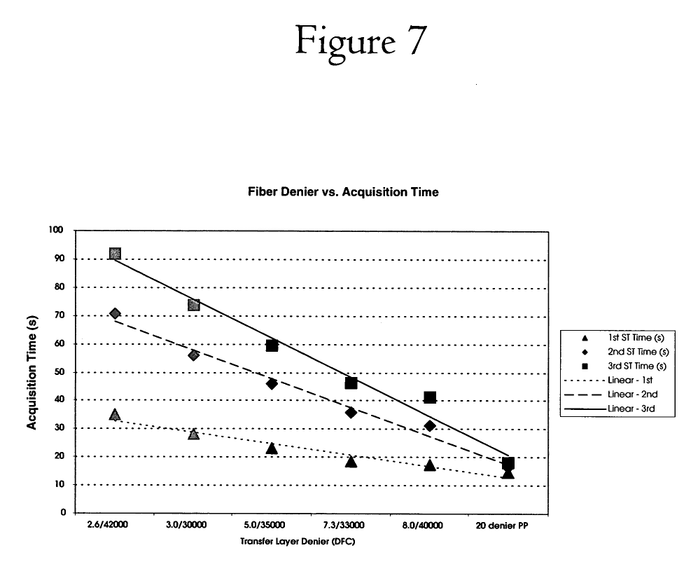

An absorbent core 34 preferably is disposed between the top sheet 30 and the back sheet 32 in at least the crotch region 16. The absorbent core 34 may extend into either or both of the front and rear regions 12, 14. Although the absorbent core 34 depicted in FIG. 1 has a substantially rectangular shape (absorbent core 34 is outlined by dotted lines), other shapes may be used, such as a “T” shape, an hourglass shape, or the shape shown in FIG. 7. The shape of the absorbent core 34 may be selected to provide the greatest absorbency with a reduced amount of material. The absorbent core may be associated with the top sheet 30, back sheet 32, or any other suitable part of the garment 10 by any method known in the art, in order to fix the absorbent core 34 in place.

The absorbent core 34 may be made from any suitable material or materials known in the art. Examples of suitable materials for use as the absorbent core 34 include creped cellulose wadding, absorbent foams, absorbent sponges, super absorbent polymers, absorbent gelling materials, fiberized cellulose, fluff pulp having tissue or synthetic materials between the absorbent core 34 and the top sheet 30 or any equivalent material or combination of materials. The size and capacity of the absorbent material should correspond to the application, for example, an incontinent brief for an adult may require a larger absorbent core than a diaper for a child. Zoned absorbency may also be used, if desired. For example, more absorbent capacity may be located in particular regions of the garment 10 depending on the gender of the intended wearer. The invention is not intended to be limited to any specific materials for use in the absorbent core 34.

In a preferred embodiment, the absorbent core 34 comprises super absorbent polymer distributed within a fibrous structure. Absorbent cores of this type are known in the art, and exemplary absorbent cores are described in U.S. Pat. Nos. 5,281,207, and 6,068,620 issued to Chmielewski et al., and U.S. Pat. No. 5,863,288, issued to Baker, the disclosures of which are herein incorporated by reference in their entirety.

Additional sublayers, transfer layers, acquisition layers, tissue wraps, and the like also may be incorporated into or otherwise associated with the absorbent core 34. Such layers may be provided to assist with transferring fluids to the absorbent core 34, handling fluid surges, preventing rewet, containing absorbent material, improving core stability, or for other purposes. For example, a substantially rectangular, preferably non-woven, sublayer (not shown), having a basis weight of about 0.1-2 oz., preferably about 0.4-0.6 oz., may overlay absorbent core 34. Those skilled in the art are capable of selecting materials, dimensions, and locations for such layers without undue experimentation.

A fluid transfer layer 40 preferably is disposed between the top sheet 30 and the absorbent core 34. The fluid transfer layer 40 preferably extends from the front region 12, through the crotch 16, and into the rear region 14, and it typically corresponds substantially to the shape of the absorbent core 34, and preferably is a little smaller. It is preferred that the fluid transfer layer 40 be approximately the same width as, or narrower than, the absorbent core 34.

The preferred embodiments of the absorbent article of the invention include a fluid transfer layer 40 comprising at least tow fibers. For example, the invention includes an absorbent article including a top sheet 30, a back sheet 32, an absorbent core 34 disposed between the top sheet and the back sheet, and a fluid transfer layer 40 disposed between the topsheet and the absorbent core. The invention also includes a method of making an absorbent article that includes providing a top sheet material and a back sheet material. The method also include preparing an absorbent core and disposing the absorbent core between the top sheet material and the back sheet material. The method further includes preparing a fluid transfer layer comprised of tow fibers, and disposing the transfer layer between the top sheet material and the absorbent core.

It is preferred in the present invention that the fluid transfer layer 40 comprised at least of tow fibers, when placed in an absorbent article 10, provide an absorbent article having a third insult strikethrough time of less than about 35 seconds, more preferably less than about 30 seconds, even more preferably, less than about 25 seconds, more preferably less than about 20 seconds, even more preferably less than about 19 seconds, and most preferably less than about 18.5 seconds.

It also is preferred in the present invention that the fluid transfer layer 40 comprised at least of tow fibers, when placed in an absorbent article 10, provide an absorbent article having a third insult rewet value of less than about 30 grams, more preferably less than about 25, even more preferably less than about 22, more preferably less than about 20, even more preferably less than about 16 grams, even more preferably less than about 15.5 grams, more preferably less than about 15 grams, even more preferably less than about 14 grams, and most preferably less than about 13.5 grams.

It is preferred in the present invention to characterize the absorbent articles by their third insult strikethrough and rewet values. This is believed to be because many materials will have comparable first insult strikethrough and rewet values, but materials that prevent leakage will differentiate from the other materials at the second and third insult values. In addition, first insult strikethrough and rewet data does not provide much information about an absorbent article in a stressed condition (e.g., at the upper end of the capability of the article). Thus, first insult strikethrough and rewet values are not necessarily good predictors of materials that will have good third insult strikethrough and rewet. In addition, it is not practical to change an absorbent article after the first insult, and in the evening, three or more insults typically occur prior to changing the absorbent article. Consequently, absorbent articles having good third insult strikethrough and rewet will be more suitable for extended use absorbent articles. Moreover, it is believed that absorbent garments that have low third insult rewet values keep the skin dryer immediately after use, and since dry skin helps keep the skin healthy, consumers recognize low rewet as a benefit. Finally, it is believed that absorbent garments having low third insult strikethrough can improve the leakage performance of the absorbent article.

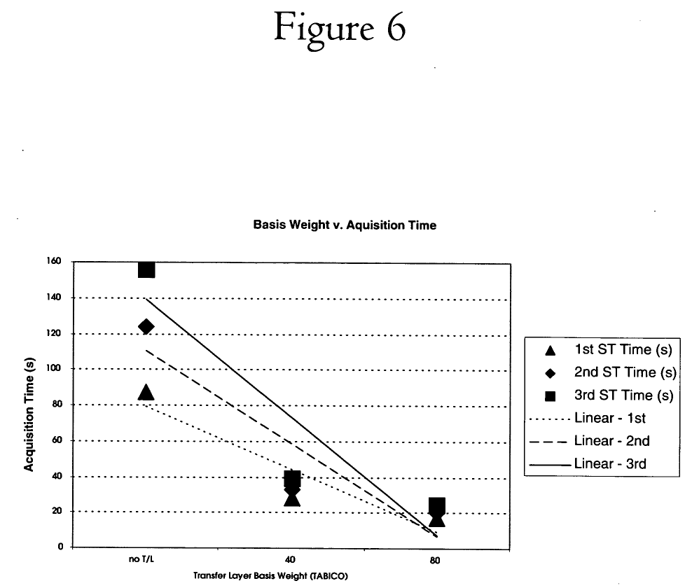

By carrying out the examples described in more detail below in the examples section, the present inventors have recognized some linear trends in absorbent article properties, when certain tow materials are used to form the fluid transfer layer 40. For example, FIG. 6 shows the relationship between the transfer layer basis weight and the strikethrough, or acquisition time. As shown in FIG. 6, the acquisition time generally decreases as the basis weight of the fluid transfer layer increases. FIG. 7 reveals that higher fiber deniers for tow fibers yields improved acquisition times. It therefore is preferred in the invention to use tow fibers having fiber deniers above about 2.0, preferably above about 3.5, more preferably above about 5.0, even more preferably above about 8.0, and most preferably from about 10 to about 50

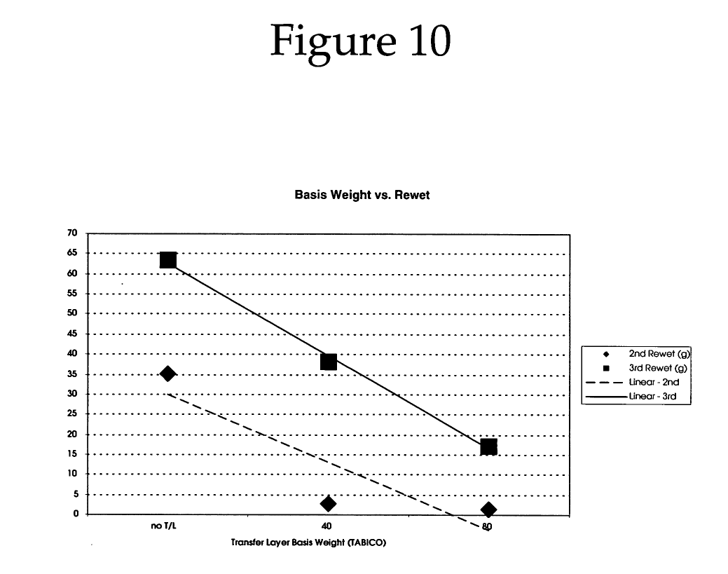

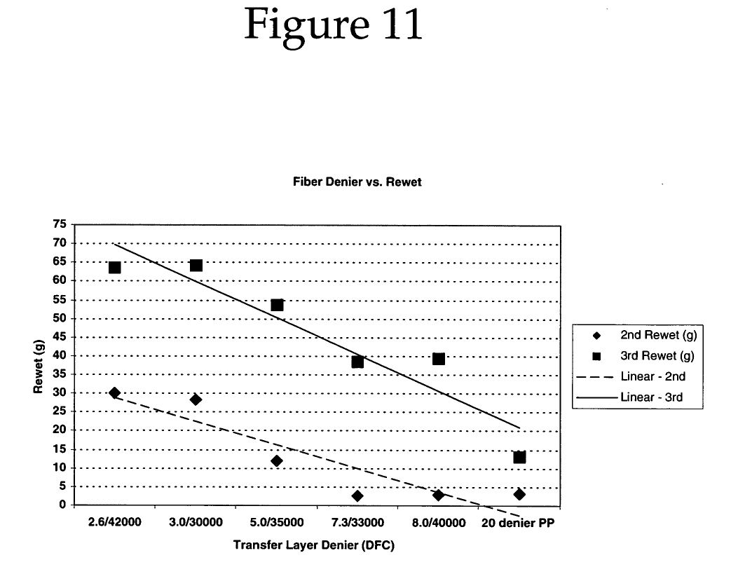

FIGS. 10 and 11 show similar relationships for rewet values. FIG. 10 reveals the same general trend observed with the strikethrough time in that the rewet values generally decrease as the basis weight of the fluid transfer layer 40 increases. The 40 and 80 gram/m² basis weight transfer layers were formed from the same known commercially available material (the 80 gram/m2 material was simply two of the 40 g/m² materials used together), as described in more detail in the examples. FIG. 11 illustrates that the rewet values generally decrease as the fiber denier of the tow fibers increases. Again, the preferred denier tow fiber useful in forming the fluid transfer layer 40 of the present invention falls within the ranges described above.

The fibrous component of the fluid transfer layer 40 of the invention preferably is comprised of at least tow fibers, and most preferably is a crimped tow of cellulose acetate, polypropylene, polyester, or mixtures thereof. Before making the fluid transfer layer 40 that includes a tow fiber, the tow fiber typically is unwound and opened, and then fed to the absorbent article manufacturing apparatus. Skilled artisans are aware of techniques available to open tow fibers and form the opened fibers into a fibrous mass. In addition, the fibrous component of the fluid transfer layer 40 may include a low-density roll good made in a separate process. Still further yet, the fibrous component could also include a carded web formed on-line. Optionally, it is advantageous to introduce from about 1-5% of a thermally bondable fiber into the fibrous component of the fluid transfer layer 40 for wet strength and stability in use. In addition to the tow material used as the fibrous component in fluid transfer layer 40, other fibrous components also may be used.

In accordance with the present invention, the fluid transfer layer 40 preferably comprises a tow fiber, and preferably, a continuous crimped filament tow. This fiber structure has high structural integrity, and as such, is distinct from a matrix of discontinuous fibers described as fluff, or fluff pulp that are conventional in the prior art. The high structural integrity enables the production of stronger webs than those formed from discontinuous fibers, which in turn are believed to enable the production of thinner absorbent articles. In addition, the use of such fibers enables the production of low density absorbent articles, when compared to conventional absorbent articles.

The tow fiber can be any continuous or discontinuous thermoplastic filament tow fiber that is capable of being opened and used in combination with SAP in an absorbent core. Preferably, polypropylene and cellulose ester tow is used as the fibrous material in central fibrous layer 284. Non-limiting examples of suitable cellulose esters include cellulose acetate, cellulose propionate, cellulose butyrate, cellulose caproate, cellulose caprylate, cellulose stearate, highly acetylated derivatives thereof such as cellulose diacetate, cellulose triacetate and cellulose tricaproate, and mixtures thereof such as cellulose acetate butyrate. A suitable cellulose ester will include some ability to absorb moisture, (but absorptive capacity is not necessarily required), preferably is biodegradable, and is influenced not only by the substituent groups but also by the degree of substitution. The relationship between substituent groups, degree of substitution and biodegradability is discussed in W. G. Glasser et al, BIOTECHNOLOGY PROGRESS, vol. 10, pp. 214-219 (1994).

Continuous filament tow useful in the present invention is beneficially moisture-absorbent and biodegradable. Accordingly, cellulose acetate and polypropylene tow is typically preferred for use in the invention. Typically, the denier per fiber (dpf) of the tow fiber will be in the range of about 2 to 45, preferably about 3 to 40, more preferably from about 5 to about 35, even more preferably, from about 8 to about 30, and most preferably about 15 to about 25. Total denier may vary within the range of about 20,000 to 60,000, more preferably from about 25,000 to about 50,000, and most preferably from about 30,000 to about 40,000, depending upon the process used.

It is particularly preferred in the invention to use tow having crimped filaments. Tow materials having crimped filaments are typically easier to open. More crimp is typically better, with in excess of about 20 crimps per inch usually being preferred. Continuous filament, cellulose ester tow having crimped filaments with about 25 to 40 crimps per inch, is commercially available from Hoechst Celanese Corporation, Charlotte, N.C.

It is preferred in the present invention that the tow fibers in fluid transfer layer 40 have an average length generally about the same length as the fluid transfer layer. Typically, the tow is a continuous filament that is cut to length during manufacture of the core. The average diameter of the tow fibers typically is expressed as the cross sectional area of the fibers, although the width of the fibers preferably is within the range of from about 50 to about 200 mm, more preferably from about 75 to about 150 mm, and most preferably from about 85 to about 120 mm. The cross sectional area is based on the denier and density of the fibers. For example, the denier per foot (dpf) and density (typically an acetate polymer density is about 1.32 g/cm³), can be used to calculate the cross sectional area. For example, a 3.0 dpf acetate polymer fiber has a cross sectional area of about 2.525×10⁻⁶ cm².

It is preferred in the invention to use relatively coarse fibers having a relatively higher basis weight to provide improved strikethrough and rewet values. The basis weight of preferred fluid transfer layers 40 of the present invention ranges anywhere from about 20 to about 200 g/m², more preferably from about 50 to about 100 g/m², and most preferably from about 70 to about 90 g/m².

If desired, a fluid transfer layer 40 of multiple layer thickness, may be provided. To this end, the tow may be, for example, lapped or crosslapped in accordance with conventional procedures. In this way, a layer of material of a desired weight and/or thickness may be provided. The specific weight or thickness will depend upon factors including the particular end use.

In addition to the tow fibers, the fluid transfer layer 40 may optionally, include from about 1-10%, preferably about 5%, by weight of thermally bondable synthetic fibers to impart additional wet strength to the layer. This will improve the stability of the layer during use of the diaper. The preferred synthetic fibers are polyolefin/polyester fibers and polyester/polyester bicomponent fibers.

It has been known to prepare absorbent cores comprised of cellulose acetate tow or other polymeric fibers and SAP, as described in H1565, and U.S. Pat. Nos. 5,436,066, and 5,350,370, the disclosures of each of which are incorporated by reference herein in their entirety. It was conventional to add tackifying agents, specific size fibers, or specific fibers in combination with fluff, in order to prepare the absorbent core and immobilize the SAP particles.

Referring back to FIG. 1, the crotch region 16 may also include mechanical sealing devices to provide the garment 10 with a leak-proof fit around the wearer. In a preferred embodiment, the crotch edges 20 each have one or more elastic gathers 36 placed along them to help the crotch edges 20 contract about the contours of the wearer's body, thereby providing a leak-proof seal. Such gathers 36 are known in the art, and are disclosed, for example, in U.S. Pat. No. 5,830,203, issued to Suzuki et al., which is herein incorporated by reference in its entirety in a manner consistent with the present invention. The gathers 36 may extend into the rear waist band 14, and may extend as far as the front longitudinally distal edge 18.

In another preferred embodiment, standing leg gathers 38 are disposed on the topsheet 30. Standing leg gathers 38 are strips of material that rise vertically from the surface of the topsheet 30 to provide additional sealing to the garment 10. The standing leg gathers 38 may extend across all or part of the garment 10 along its longitudinal axis 100. Typically, one standing leg gather 38 is located on either side of the absorbent core 34. The standing leg gathers 38 may be made from folded portions of the topsheet 30 or backsheet 32, or may be made from additional strips of material. Each standing leg gather 38 may be equipped with one or more elastic elements to help seal the gather to the wearer's body. Standing leg gathers are known in the art, and disclosed in U.S. Pat. No. 5,292,316, issued to Suzuki, which is herein incorporated by reference in its entirety in a manner consistent with the present invention.

Any suitable elastic material may be used for the gathers 36 and the standing leg gathers 38. Preferably, the elastic material can be stretched to between more than 10% to 300% of its original length without losing its resilience. The elastic material used for the gathers 36 and standing leg gathers 38 may comprise an elastic film, a multidirectional elastic aggregate such as elastic webbing, netting, or scrim elastic, such as FLEXCEL™ Elastic Nonwoven Laminate, available from Kimberly-Clark Corporation, headquartered in Neenah, Wis., or strands or bands of suitable elastic materials, such as natural or synthetic rubber, urethane elastomers, spandex, LYCRA and elastic polymers. The elastics materials may be attached to the garment 10 in any of several ways known in the art. For example, the elastic materials may be ultrasonically bonded, heat/pressure sealed using a variety of bonding patterns, or glued to the diaper 10 using a variety of adhesives. Other performance enhancing devices, such as pockets, baffles, and openings in the topsheet also may be used with the present invention.

The absorbent article is optionally a diaper, incontinent brief, training pant, diaper holder, diaper liner, sanitary napkin, hygienic garment or combinations thereof. Diapers may include daytime diapers, nighttime diapers, long-term wear diapers, travel diapers, swimming diapers, daytime/nighttime diapers, male diapers, female diapers, unisex diapers, active diapers, seasonal diapers, cold weather diapers, warm weather diapers, medicated diapers, inactive diapers for newborns, specialty diapers for ill children having a higher incidence of runny BM, or combinations thereof.

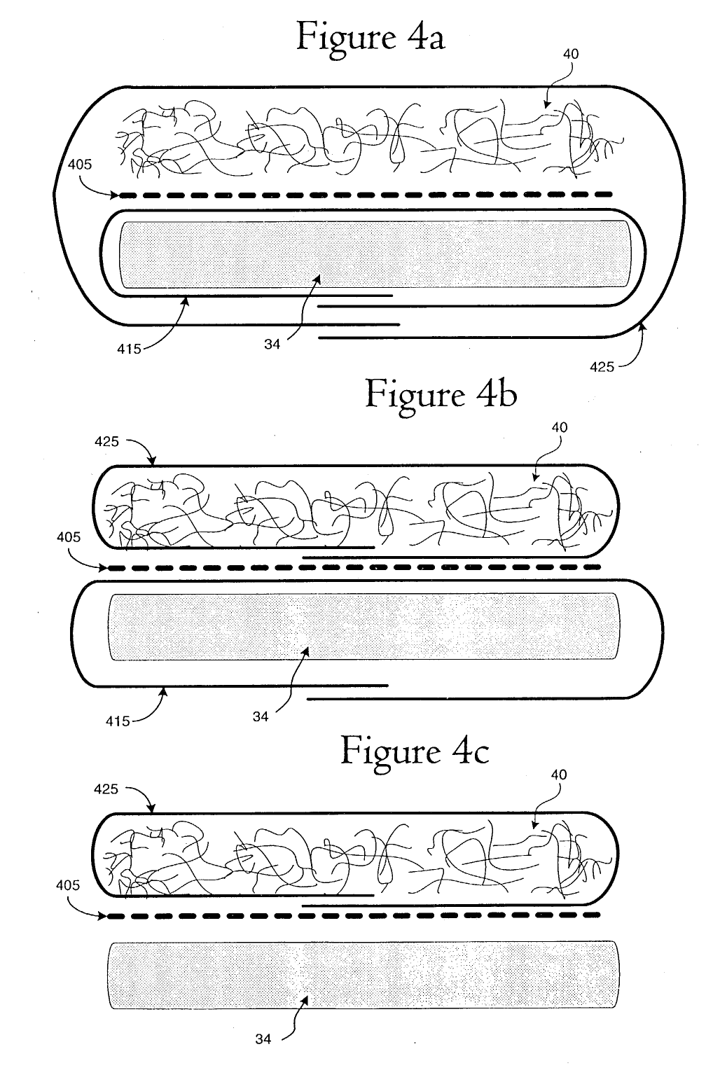

The fluid transfer layer 40 may be configured in the absorbent article as shown in FIG. 1, although it is preferred that the longitudinal dimension of the fluid handling layer 40 be shorter than that of the underlying absorbent core 34. Preferably, the fluid transfer layer 40 covers the insult area of the absorbent core 34, and is primarily disposed, for example, in the front half of the garment near the front region 12. The particular manner in which the fluid handling layer is attached to the absorbent garment 10 is not critical to the present invention. Various configurations are illustrated in FIG. 3a, FIG. 3b, FIG. 4a, FIG. 4b, and FIG. 4c.

In FIG. 3a, the fluid transfer layer 40 is attached to an absorbent core wrapped in a wrapping 415 by use of optional adhesive 405. The tow fibers can be opened in-line and directly deposited on the wrapped absorbent core 34. Adhesive 405 may or may not be used, and any adhesive can be used to adhere the tow to the core. In FIG. 3b, the fluid transfer layer 40 is attached to an unwrapped absorbent core 34 by use of optional adhesive 405.

FIGS. 4a, 4b, and 4c illustrate various embodiments whereby the fluid transfer layer 40 is wrapped with a wrapping material 415, 425. In FIG. 4a, the absorbent core 34 and fluid transfer layer 40 are wrapped with the same wrapping 415 that envelops both components of the absorbent article. FIG. 4b illustrates an embodiment whereby the absorbent core 34 is wrapped with wrapping material 415, and the fluid transfer layer 40 is wrapped with wrapping material 425. FIG. 4c illustrates an embodiment whereby only the fluid transfer layer 40 is wrapped with wrapping material 425.

The particular material used for wrapping material 415, 425 is not limited in the invention, and two different materials may be used such that the wrapping includes an upper and lower layer. The upper and lower layer preferably encase the absorbent core 34 and/or fluid transfer layer 40, or both, and may form the upper and lower tissue layers of the completed garment, but may also form the top sheet and back sheet of the absorbent garment, or any other layers. The upper and lower layers preferably are wider than the respective components in which they encase, and their side portions preferably are sealed to one another by bonding, by crimping or by both to prevent release of opened tow and/or particles of SAP. In addition, as shown in the figures, the upper and lower layers preferably are comprised of the same material folded over onto itself, and only the open end is sealed by crimping or bonding. The absorbent core 34, optionally comprising the upper and lower layers, as well as the fluid transfer layer 40, also optionally comprising the upper and lower layers, may be further processed as it is conveyed through the assembly line for inclusion into absorbent garments. For example, the absorbent core 34 or fluid transfer layer 40, or both, may be severed into individual absorbent cores, and the severed ends may be crimped or bonded or both.

Crimping, bonding or both can be performed on the absorbent core 34, the fluid transfer layer 40, or both using conventional means. For example, the lateral side edges, and longitudinal edges can be sealed together by intermittent or continuous application of adhesive to the respective portions of the upper and lower layers using any device capable of applying adhesives to a continuous moving web of material. The lateral and/or longitudinal edges then can be pressed together to form a seal. The seal also can be formed ultrasonically, or the respective edges (lateral and/or longitudinal) can be crimped using crimping rollers or any other crimping device known to those having ordinary skill in the art. Using the guidelines provided herein, those skilled in the art will be capable of sealing the lateral and/or longitudinal edges of absorbent core 34, fluid transfer layer 40, or both using bonding, crimping, or both.

Any material may be used for wrapping material 415, 425. When the absorbent core 34 is wrapped with wrapping material 415, it is preferred that the upper portion or upper layer of the wrapping material 415 be fluid pervious, and the lower portion or lower layer be fluid impervious. Different materials may be used, or the same material used and treated to effect the desired fluid perviousness. Tissue layers, carded layers, non-woven materials, and other cellulose-based layers can be used as wrapping material 415, 425.

The invention also relates to a method of making an absorbent article that includes providing a top sheet material 30 and a back sheet material 32. The method also includes preparing an absorbent assembly by preparing an absorbent core 34 and disposing on top of the absorbent core a fluid transfer layer 40. The method is completed by disposing the absorbent assembly (e.g., core 34 and transfer layer 40) between the top sheet 30 and the back sheet 32. The embodiments illustrated in FIGS. 3a, 3b, 4a, 4b, and 4c can be prepared in accordance with the method simply by wrapping the respective components, e.g., absorbent core 34 or fluid transfer layer 40, with wrapping material 415, 325, respectively, and then preparing the absorbent assembly. The embodiment of FIG. 4a can be prepared by disposing the fluid transfer layer 40 on top of absorbent core 34, and then wrapping the assembly with wrapping material 415.

The method of making the fluid transfer layer 40 containing the tow materials can be prepared in any manner known in the art for making fluid transfer, or fluid acquisition layers. Preferably, the tow fibers are opened using a tow-opening apparatus, as are known in the art. Suitable apparatus for opening a tow, and forming a layer out of the tow, are described in U.S. Pat. Nos. 6,253,431 and 6,543,106, the disclosures of each of which are incorporated by reference herein in their entirety. The tow fibers can be cut to length using a heated cutting knife to seal the ends of the fibers and to assist in maintaining the integrity of the layer. In addition, the tow fibers can be bonded to one another in any bonding pattern (e.g., a waffle pattern), to maintain the integrity of the layer, as described, for example, in U.S. Pat. Nos. 4,360,022, 6,245,961, 6,417,427, and 6,511,566. Using the guidelines provided herein, those skilled in the art will be capable of opening a tow material, forming a fluid transfer layer from the opened tow, and disposing the fluid transfer layer between an absorbent core and top sheet of an absorbent article.

The invention now will be explained with reference to the following non-limiting examples.

### EXAMPLES

The test methods used to measure the strikethrough and rewet values were as follows:

Sample Preparation:

Absorbent articles were prepared for testing in accordance with the following procedure. Conventional absorbent articles were constructed using known apparatus and known materials, including a topsheet 30, backsheet 32, and absorbent core 34. The conventional fluid transfer layer used herein for comparison purposes was a 40 g/m² carded thermal bond material commercially available as TABICO from Polymer Group, Inc. North Charleston, S.C. The conventional diapers were Stage 3 diapers, available from Tyco Healthcare, King of Prussia, Pa. The conventional articles were de-constructed by cutting the top sheet 30 at its longitudinal ends. Fluid transfer layers were prepared by forming the layer with various tow materials, as described in the examples, or by using the conventional TABICO materials. The fluid transfer layers prepared using the tow materials were prepared by processing the tow materials to open the tow, and then gluing the tow fibers onto a TAD tissue, which was Cellu-tissue grade 4214-30, which is commercially available from Interlake Paper, Inc., St. Catharines, Ontario, Canada. The fluid transfer layers 40 then were placed on top of the core, and the top sheet re-sealed.

The reconstructed absorbent articles then were subjected to the following testing procedures.

Testing Procedures

### Measuring Strikethrough and Rewet

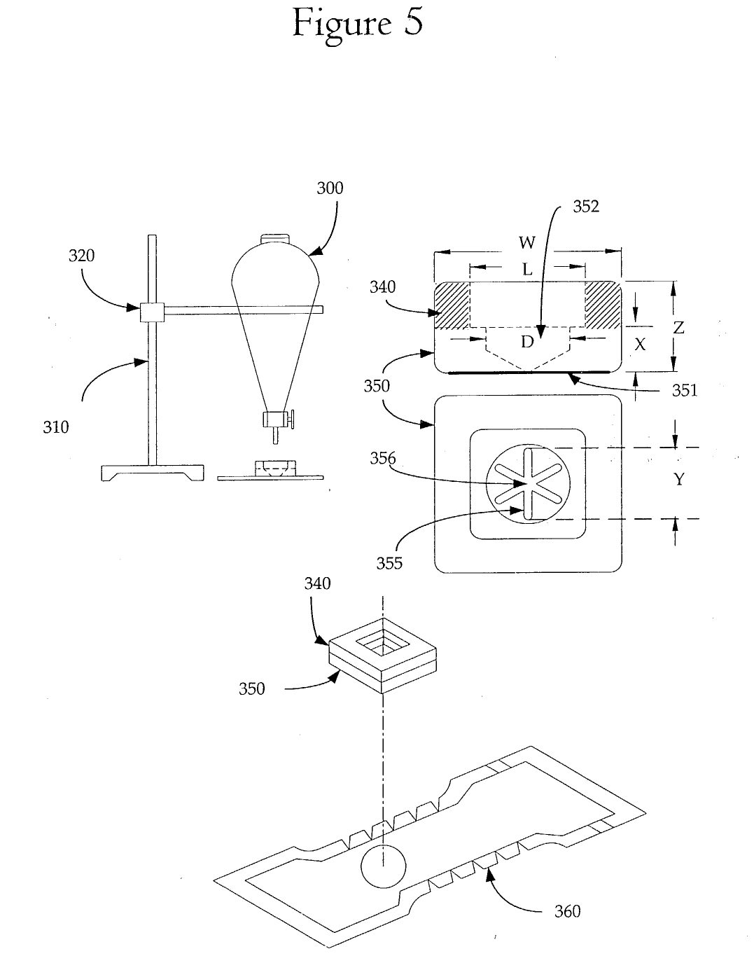

The apparatus used to measure strikethrough and rewet generally is shown in FIG. 5. The materials used include a burette (not shown) with discharge valve, preferably a 250 ml burette, and a separatory funnel 300, preferably a 150 ml separatory funnel having a 6-7 ml/second stopcock. The apparatus further included a stand 310 with a clamp 320, and a strikethrough plate 350, which included a Plexiglas cover plate 340 (4″×4″). Other materials used in the strikethrough and rewet test included a 0.5 psi weight, (2.5″×2.5″) Filter paper, Fisher Brand P8 filter paper cut to the appropriate size, synthetic urine in the form of a 0.9% NaCl solution with 0.0025% Triton X-100 surfactant, a Plexiglas coverplate 4″×4″×¼″, a timer, a balance with 0.01 gram accuracy, and a stretch board with clamps 360. The diaper chassis was prepared on a conventional diaper line without topsheet glue and without a transfer layer to enable the sample preparation described above.

As shown in FIG. 5, the strikethrough plate has specifically designed parameters. The strikethrough plate used to measure the strikethrough and rewet properties of the diapers meets the requirements set forth in edana Liquid Strike-Through Time Section 150.4-99, February 1999. Specifically, the strikethrough plate was constructed of a transparent acrylic sheet about 20 mm thick, which was placed on top of the strikethrough plate, having a thickness of about 17.8 mm. The strikethrough plate was fitted with corrosion-resistant electrodes 351 consisting of about 1.6 mm diameter platinum or stainless steel wire set in grooves of cross section of about 4.0 mm×7.0 mm cut into the base of the plate and fixed with quick-setting epoxy. The strikethrough plate 350 had an outlet opening at the top of the 17.8 mm deep bore 352, having a diameter D of a little more than about 25 mm. Thus, the values for X and D in FIG. 5 preferably are 17.8 mm, and 25 mm, respectively, although those skilled in the art will appreciate that the particular dimensions of D and X can vary. The bottom of the bore preferably had a center circular hub 356 with a diameter of about 1.9 mm with 6 symmetrically positioned spokes 355 extending about 11 mm from the hub. The value for Y in FIG. 5 therefore was about 22.225 mm, or about twice the length of each symmetrically positioned spoke 355. The slope of the bottom of bore 352 was about 25° from level.

The values for L, W, and Z in FIG. 5 also may vary, as will be appreciated by one of ordinary skill in the art. It was preferred that L be on the order of about 30 mm and W could be up to about 125 mm, but preferably was 70 mm. The overall thickness of the strikethrough plate, including strikethrough plate 350 and baseplate 340, designated by “Z” in FIG. 5, preferably was about 37.8 mm, whereby the baseplate 340 was about 20 mm thick.

The samples preferably were prepared for the strikethrough and rewet test by selecting 3 mean weight diapers, prepared by the sample preparation technique described above, that were free of lumps, creases, and wrinkles. The diapers were weighed to the nearest 0.1 g. The insult point then was marked, which typically was positioned about ½ the distance from the midpoint of the diaper to the end point of the absorbent core. The insult point also was at the midpoint of the width of the absorbent core.

The synthetic urine was prepared by weighing approximately 5 g of Triton X-100 into a clean, 2000 ml flask. Then, about 18 g NaCl was weighed and transferred into the same 2000 ml flask container with the Triton X-100, and diluted with de-ionized water to 2000 ml liter. The solution then preferably was stirred. The test solutions were discarded if not used within seven days, or if the percent saline was not about 0.9% by weight, as measured using a refractometer.

Strikethrough and rewet then were tested in accordance with the following procedure.

The diaper was stretched onto a stretch board with clamps 360, making sure there were no bumps or wrinkles in the diaper (see, FIG. 5). The basis weight of the transfer layers were determined on one meter strips of the transfer layer, where the basis weight was recorded in grams per square meter. The transfer layer was cut to about a 360 mm length by 70 mm width, taped to a tissue layer, and pulled through the diaper chassis after cutting the top sheet at both longitudinal ends. Then, the insult point on the diaper was marked, and the strikethrough plate 350 was centered on the insult point.

Referring again to FIG. 5, the separatory funnel 300 was filled with 100 ml of test solution, and the tip of the separatory funnel 300 was centered a few mm above the strikethrough plate 350. The separatory funnel valve then was opened and the timer started at the exact same time, keeping the cavity of the strikethrough plate 352 completely full with solution to maintain constant pressure. When the complete 100 ml solution had been absorbed into the diaper, the timer was stopped, and the strikethrough value recorded in seconds.

After recording the strikethrough time, a 0.5 psi weight was placed onto the insult point, and the timer was set for 10 minutes. Then, about 18 g of filter paper was weighed, and its weight recorded to the nearest 0.1 gram. After about 10 minutes had elapsed, the 0.5 psi weight was removed, the filter papers were placed on the insult point, the 0.5 psi weight was replaced, and the timer again set for 10 minutes. After 10 minutes had elapsed, the weight and the filter papers were removed, and the filter papers weighed, and the weight recorded to the nearest 0.1 gram. This value was the rewet value.

This procedure from first striking the insult point with the 100 ml of solution, until the filter papers were weighed consisted of an insult. Since this was the first time, it was denoted hereinafter as the first insult. The procedure then was repeated on the same diaper sample for a second insult. For the second insult, however, about 50 grams of filter paper were used. The procedure then can be repeated on the same sample a third time for a third insult. For the third insult, however, about 72 grams of filter paper were used.

The rewet was calculated by taking the difference in weight of the filter paper weighed before being placed on the diaper and after ten minutes had elapsed with the weight placed thereon. The rewet values were designated as the first insult rewet, second insult rewet, and third insult rewet, respectively. The strikethrough value was the amount of time, in seconds, taken for 100 ml of solution to completely absorb into the diaper. The strikethrough and rewet values were taken for all three samples, and the reported values in the table below (and in FIGS. 6-11) represent the average for all three samples.

### EXAMPLES

Samples were prepared as described above for various transfer layers. Control A in the table below was a diaper with no transfer layer. Control B was the same diaper with a 40 g/m² carded thermal bond material commercially available as TABICO from Polymer Group, Inc. North Charleston, S.C. Control C was the same diaper as Control B, except that it included two 40 g/m² carded thermal bond material transfer layers, each commercially available as TABICO from Polymer Group, Inc. North Charleston, S.C.

The remaining samples listed in Table 1 below were transfer layers prepared using a tow fiber. All tow fibers were cellulose acetate tow, except the product designated as 20 denier PP, which was a polypropylene tow fiber having a denier of 20. For the cellulose acetate tow fibers, the first number in the product designation denotes the denier per fiber, and the second number after the slash (“/”) denotes the total denier.

Each of the samples were subjected to three consecutive strikethrough and rewet testing procedures described above. The results are found in Table 1 below:

TABLE 1

| | Basis | | | | | | 3rd |
|---|---|---|---|---|---|---|---|
| | Wgt | 1st ST | 2nd ST | 3rd ST | 1st | 2nd Rewet | Rewet |
| Product | (gsm) | Time (s) | Time (s) | Time (s) | Rewet (g) | (g) | (g) |
| Control A | no T/L | 87.47 | 123.80 | 155.85 | 0.53 | 35.03 | 63.40 |
| Control B | 40 | 28.23 | 32.96 | 39.38 | 0.20 | 2.63 | 38.00 |
| Control C | 80 | 17.38 | 19.97 | 24.27 | 0.13 | 1.33 | 17.27 |
| 2.6/42000 | 78 | 35.12 | 70.68 | 91.95 | 0.20 | 30.10 | 63.50 |
| 3.0/30000 | 86 | 28.32 | 55.99 | 73.90 | 0.20 | 28.40 | 64.33 |
| 5.0/35000 | 72 | 23.06 | 45.82 | 59.31 | 0.20 | 12.03 | 53.80 |
| 7.3/33000 | 89 | 18.47 | 36.03 | 46.15 | 0.13 | 2.57 | 38.53 |
| 8.0/40000 | 73 | 17.49 | 31.43 | 41.38 | 0.20 | 3.03 | 39.50 |
| 20 denier PP | 80 | 14.63 | 16.59 | 18.08 | 0.30 | 3.20 | 13.10 |

The results of Table 1 are reflected in graphical form in FIGS. 6-11. FIG. 6 illustrates the relationship between basis weight and strikethrough, or acquisition time. The results in FIG. 6 are those from Control A (no T/L), Control B (40), and Control C (80). As shown in FIG. 6, the acquisition time decreases as the basis weight increases, for similar materials. A similar trend is seen for rewet values, and is shown graphically in FIG. 10. FIG. 10 also reveals that the rewet decreases as the basis weight increases. It therefore is desirable to use a transfer layer having a basis weight on the order of about 80 g/m², although conventional transfer layers having a basis weight of about 40 g/m² provide suitable results.

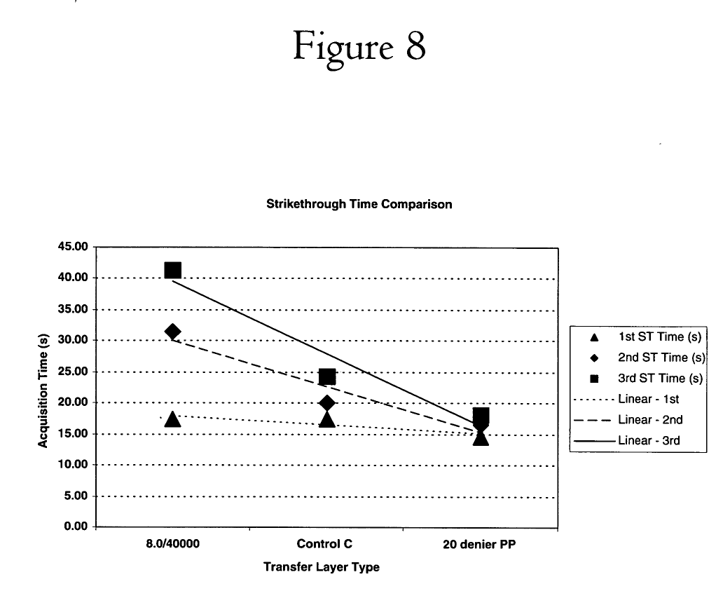

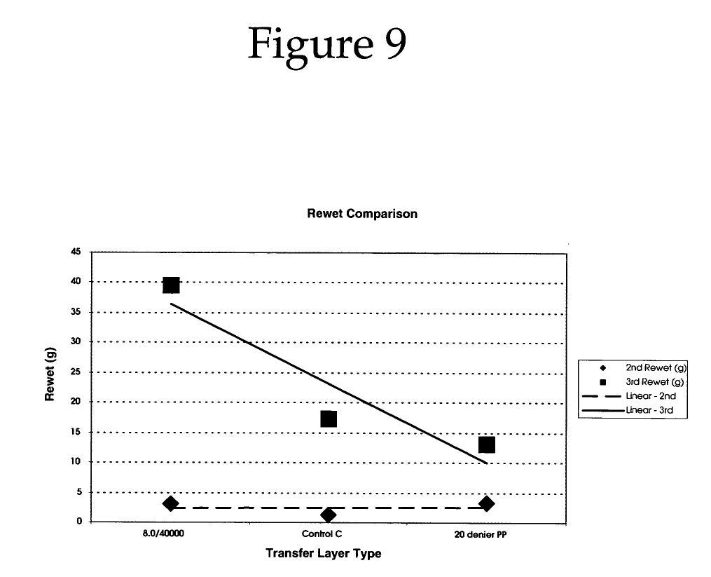

To provide a reasonable comparison between various tow fibers, transfer layers having comparable basis weights were compared, while varying the type of tow fiber (e.g., cellulose acetate vs. polypropylene), and the denier of the fiber. FIG. 7 reveals that as the denier of the fiber increases, the acquisition time decreases, with the best results seen for the example using a 20 denier polypropylene tow fiber. FIG. 8 reveals that the 20 denier polypropylene tow fiber provided superior acquisition rates, when compared to a modified conventional transfer layer (modified in the sense that two transfer layers having a basis weight of 40 g/m²) were superimposed upon each other. A similar affect is illustrated in FIG. 9 with respect to rewet. Finally, FIG. 11 demonstrates that, as the denier of the fiber increases, the rewet decreases, with the best results seen for the example using a 20 denier polypropylene tow fiber.

Other embodiments, uses, and advantages of the invention will be apparent to those skilled in the art from consideration of the specification and practice of the invention disclosed herein. The specification should be considered exemplary only, and the scope of the invention is accordingly intended to be limited only by the following claims.

What is claimed is:

1. An absorbent article comprising:
a top sheet;
a back sheet;
a absorbent core disposed at least partially between the top sheet and the back sheet; and
a fluid transfer layer disposed at least partially between the absorbent core and the top sheet, wherein the fluid transfer layer is comprised at least in part of tow fibers,
and wherein absorbent article has a third insult strikethrough time of less than about 35 seconds, and a third insult rewet value of less than about 30 grams.

2. The absorbent article of claim 1, whereby the article has a first waist region, a second waist region longitudinally opposed to the first waist region, and a crotch region between the first and second waist regions, the article further comprising at least one fastening element attached to a lateral edge of the first waist region; and
one or more target devices attached to the article in the second waist region, where at least one fastening element and the one or more target devices are capable of attaching to one another, the one or more target devices being located so that the first waist region and second waist region of the garment may be joined to one another to secure the garment on a wearer.

3. The absorbent article of claim 2, further comprising elastic leg gathers comprising one or more elastic materials disposed adjacent a lateral edge of the crotch region, and standing leg gathers disposed on the top sheet adjacent the lateral edge of the crotch region.

4. The absorbent article of claim 2, wherein the at least one fastening element comprises a hook portion of a hook and loop fastener and the one or more target devices comprise the loop portion of a hook and loop fastener.

5. The absorbent article of claim 2, wherein the at least one fastening element is an adhesive tape and the one or more target devices comprise a tape receiving surface.

6. The absorbent article of claim 2, wherein the at least one fastening element is comprised of a pair of laterally extending tabs disposed on the lateral edges of the first waist region, whereby the laterally extending tabs each include at least one fastening element.

7. The absorbent article of claim 1, wherein the fluid transfer layer comprises tow fibers selected from the group consisting of cellulose acetate fibers, polypropylene fibers, polyester fibers, rayon fibers, LYOCELL fibers, polyacrylonitrile fibers, cotton fibers, cotton linter fibers, and mixtures thereof.

8. The absorbent article of claim 7, wherein the tow is a cellulose ester tow or a polypropylene tow.

9. The absorbent article of claim 8, wherein the tow is a cellulose acetate tow.

10. The absorbent article of claim 8, wherein the to is a polypropylene tow.

11. The absorbent article of claim 1, wherein the absorbent article has a third insult strikethrough time of less than about 30 seconds and a third insult rewet value of less than about 25 grams.

12. The absorbent article of claim 11, wherein the absorbent article has a third insult strikethrough time of less than about 20 seconds and a third insult rewet value of less than about 20 grams.

13. The absorbent article of claim 1, wherein the tow fibers have a denier per fiber (dpf) within the range of from about 2 to about 45.

14. The absorbent article of claim 13, wherein the tow fibers have a dpf within the range of from about 8 to about 30.

15. The absorbent article of claim 13, wherein the tow fibers have a dpf within the range of from about 15 to about 25.

16. The absorbent article of claim 10, wherein the polypropylene tow has a dpf of about 20.

17. The absorbent article of claim 1, wherein the tow fibers have a total denier within the range of from about 20,000 to about 60,000.

18. The absorbent article of claim 17, wherein the tow fibers have a total denier within the range of from about 25,000 to about 50,000.

19. The absorbent article of claim 17, wherein the tow fibers have a total denier within the range of from about 30,000 to about 40,000.

20. A method of making an absorbent article comprising:
a) preparing a top sheet and a back sheet;
b) preparing an absorbent core;
c) disposing the absorbent core at least partially between the top sheet and the back sheet;
d) preparing a fluid transfer layer comprised at least partially of tow fibers; and
e) disposing the transfer layer between the top sheet material and the absorbent core to form an absorbent article that has a third insult strikethrough time of less than about 35 seconds, and a third insult rewet value of less than about 30 grams.

21. The method of claim 20, wherein preparing the fluid transfer layer comprises opening the tow fibers and forming a layer of tow fibers.

22. The method of claim 20, wherein the fluid transfer layer comprises tow fibers selected from the group consisting of cellulose acetate fibers, polypropylene fibers, polyester fibers, rayon fibers, LYOCELL fibers, polyacrylonitrile fibers, cotton fibers, cotton linter fibers, and mixtures thereof.

23. The method of claim 22, wherein the tow is a cellulose ester tow or a polypropylene tow.

24. The method of claim 23, wherein the tow is a cellulose acetate tow.

25. The method of claim 23, wherein the to is a polypropylene tow.

26. The method of claim 20, wherein the absorbent article has a third insult strikethrough time of less than about 30 seconds and a third insult rewet value of less than about 25 grams.

27. The method of claim 26, wherein the absorbent article has a third insult strikethrough time of less than about 20 seconds and a third insult rewet value of less than about 20 grams.

28. The method of claim 20, wherein the tow fibers have a denier per fiber (dpf) within the range of from about 2 to about 45.

29. The method of claim 28, wherein the tow fibers have a dpf within the range of from about 8 to about 30.

30. The method of claim 28, wherein the tow fibers have a dpf within the range of from about 15 to about 25.

31. The method of claim 25, wherein the polypropylene tow has a dpf of about 20.

32. The method of claim 20, wherein the tow fibers have a total denier within the range of from about 20,000 to about 60,000.

33. The method of claim 32, wherein the tow fibers have a total denier within the range of from about 25,000 to about 50,000.

34. The method of claim 32, wherein the tow fibers have a total denier within the range of from about 30,000 to about 40,000.
